(19) United States
(12) Patent Application Publication
(10) Pub. No.: US 2023/0415140 A1
(43) Pub. Date: Dec. 28, 2023

(54) CATALYTIC ACTIVITY RECOVERY METHOD OF MANGANESE OXIDE CATALYST

(71) Applicant: PURESPACE INC., Busan (KR)

(72) Inventors: Jeong Se YUN, Busan (KR); Jae Sung LEE, Busan (KR); Hae Young JEONG, Busan (KR); Sun Young LEE, Seoul (KR)

(73) Assignee: PURESPACE INC., Busan (KR)

(21) Appl. No.: 18/036,648

(22) PCT Filed: Nov. 12, 2020
(86) PCT No.: PCT/KR2020/015852

§ 371 Date: May 12, 2023

(51) Int. Cl. B01J 38/12; B01J 23/34; B01J 23/92; B01J 38/02; B01D 53/86; B01J 37/03; B01J 37/10; B01J 37/02; A61L 9/015

(52) U.S. Cl. CPC B01J 38/12; B01J 23/34; B01J 23/92; B01J 38/02; B01D 53/8675; B01D 53/864; B01J 37/033; B01J 37/10; B01J 37/0215; A61L 9/015; B01D 2257/7022; B01D 2257/106; B01D 2258/06; B01D 2251/104; B01D 2255/2073; B01D 2257/91

(57) ABSTRACT

Provided is a catalytic activity recovery method of a manganese oxide catalyst, an air-cleaning device using the same, air-cleaning system including the air-cleaning device, and an operation method of air-cleaning device by using the manganese oxide catalyst. The catalytic activity recovery method of a manganese oxide catalyst includes recovering the initial activity of a manganese Ni oxide catalyst by heating a manganese oxide catalyst which has been used to decompose ozone and of which activity is thus reduced by 10% or more compared to the initial ozone decomposition efficiency thereof, at the temperature of 80° C. to 250° C., so as to recover an ozone decomposition efficiency represented by Equation 1 to 90% or more of the initial ozone decomposition efficiency: Equation 1 Ozone decomposition efficiency (%)=[1−(concentration of ozone flowing out of the reactor)/(concentration of ozone flowing into the reactor)]×100

### TECHNICAL FIELD

The present disclosure relates to an catalytic activity recovery method of manganese oxide catalyst, an air-cleaning device using the same, an air-cleaning system using the air-cleaning device, and an operation method of the air-cleaning device using the manganese oxide catalyst.

### BACKGROUND ART

It is important to remove organic substances from the air in aspects of maintenance of the freshness of fruits and vegetables, as well as health and accident prevention through a decrease in harmful substances. This is because, after plant growth and harvest, harmful gases including ethylene, which are closely involved in the growth of crops, and harmful microorganisms such as airborne microbe, fungi, and bacteria, spread through the air.

In order to reduce or remove harmful gases including ethylene and harmful microorganisms, various attempts have been made using filters, adsorption, and chemical reactions. Among them, chemical reactions that can function with high efficiency for a long time have been utilized. The method using chemical reactions is performed based on such a mechanism that oxygen-based radicals and ozone with high oxidizing power are used to remove organic substances and the remaining ozone is removed, and then, clean air is discharged into the atmosphere. However, the lifespan of ozone decomposition catalysts, used in such a method applied in an air-cleaning device used to reduce or remove harmful gases including ethylene and harmful microorganisms may be dramatically decreased in a low temperature and high humidity environment.

Therefore, there is a need to develop an catalytic activity recovery method of new catalysts to effectively reduce or remove harmful gases including ethylene and harmful microorganisms and ozone by recovering the activity of ozone decomposition catalysts, an air-cleaning device for reducing harmful gases including ethylene and harmful microorganisms, and an air-cleaning system including the same.

### DISCLOSURE OF INVENTION

### Technical Problem

One aspect is to provide catalytic activity recovery method of a novel manganese oxide catalyst.

Another aspect is to provide an air-cleaning device using the catalytic activity recovery method.

Another aspect is to provide an air-cleaning system including the air-cleaning device.

Another aspect is to provide an operation method of an air-cleaning device using the manganese oxide catalyst.

### Solution to Problem

According to one aspect,
provided is a catalytic activity recovery method of a manganese oxide catalyst including recovering an ozone decomposition efficiency represented by Equation 1 to 90% or more of the initial ozone decomposition efficiency by heating, in an air atmosphere, a manganese oxide catalyst which has been used to decompose ozone and of which activity is thus reduced by 10% or more of the initial ozone decomposition efficiency thereof, at a temperature ranging from 80° C. to 250° C.:

Ozone decomposition efficiency (%)=[1−(concentration of ozone flowing out of the reactor)/(concentration of ozone flowing into the reactor)]×100 Equation 1

The catalytic activity recovery method may include recovering the ozone decomposition efficiency represented by Equation 1 to 95% or more of the initial ozone decomposition efficiency.

The heating may include periodical heating for 10 minutes to 10 hours in a temperature range of 80° C. to 250° C. while one cycle is set to a time period from a time point when the ozone decomposition efficiency represented by Equation 1 is reduced to less than 90% of the initial ozone decomposition efficiency to a time point when the ozone decomposition efficiency is restored to 90% or more of the initial ozone decomposition efficiency.

The heating may include periodical heating at a heating rate of 1° C./min to 10° C./min for 10 minutes to 10 hours in a temperature range of 80° C. to 250° C. while one cycle is set to a time period from a time point when the ozone decomposition efficiency represented by Equation 1 is reduced to less than 90% of the initial ozone decomposition efficiency to a time point when the ozone decomposition efficiency is restored to 90% or more of the initial ozone decomposition efficiency.

The heating may be performed using one of a planar heating element, an electric resistance heating device, a heating furnace, a heating oven, infrared-ray heating, or a microwave generating unit.

The planar heating element may include a polymer resin, a carbon material, a metal material, a ceramic material, or a composite thereof.

The manganese oxide catalyst may include a nano manganese oxide comprising at least one of α-MnO₂, β-MnO₂, γ-MnO₂, δ-MnO₂, or amorphous MnO₂.

The manganese oxide catalyst may have a shape selected from a sphere shape, an oval shape, a rod shape, a fiber shape, a sea-urchin shape, a flower shape, or a sheet shape.

The manganese oxide catalyst may include at least one of α-MnO₂ or β-MnO₂, and
the α-MnO₂ or β-MnO₂ has a nanorod shape, a nanofiber shape, a nano sea-urchin shape, or a nanoflower shape, and has an aspect ratio of 1:5 to 1:1000.

The manganese oxide catalyst may be α-MnO₂, and
the α-MnO₂ may be in the shape of a nanorod or a nano sea-urchin, and the aspect ratio thereof may be 1:100.

The manganese oxide catalyst may further include a manganese oxide doped with a transition metal of 0.01 wt % to 50 wt % based on the total weight of the manganese oxide catalyst.

The manganese oxide catalyst may further include at least one selected from metal oxide, silicon oxide, carbon nanotubes, activated carbon, graphene, or graphene oxide.

According to another aspect,
provided is an air-cleaning device using the catalytic activity recovery method of a manganese oxide catalyst.

The air-cleaning device for reducing harmful gases including ethylene and harmful microorganisms includes:
an ozone generating unit in which an ozone generating unit driven by electric energy to generate ozone is located;
an ozone decomposition unit in which at least one ozone decomposition catalyst structure for decomposing ozone generated by the ozone generating unit is located; and
a heating member for heating the ozone decomposition catalyst structure,
the ozone decomposition catalyst structure includes a support and nano manganese oxide located on at least a portion of the surface and inside of the support,
the heating member is located within the ozone decomposition unit or located separate from the ozone decomposition unit, and
allows the inflow and outflow of air in one direction.

The support may be a ceramic material, a metal material, or combination of these, in the form of a monolith or a foam.

The ozone decomposition catalyst structure may be a binder-free structure.

At least one side of the ozone decomposition catalyst structure may be in contact with a sheet, film, pad, or foil of a thermally conductive material.

The heating member may be one of a planar heating element, an electric resistance heating device, or a microwave generating unit.

The planar heating element may include a polymer resin, a carbon material, or a composite thereof, which may be in the form of a sheet or a film.

The surface heating element may be in contact with an insulating material.

The ozone generating unit may be one or more of a vacuum ultraviolet lamp, a corona discharge ozone generator, or a cold plasma ozone generator.

The air-cleaning device may maintain an ozone decomposition efficiency represented by Equation 2 to be 90% or more of the initial ozone decomposition efficiency by periodical heating, at the temperature of 80° C. to 250° C., a nano manganese oxide catalyst which has been used to decompose ozone and of which activity is thus reduced by 10% or more of the initial ozone decomposition efficiency thereof, from a time point when the ozone decomposition efficiency represented by Equation 2 is reduced to less than 90% of the initial ozone decomposition efficiency to a time point when the ozone decomposition efficiency is restored to 90% or more compared to the initial ozone decomposition efficiency, to recover the catalytic activity.

Ozone decomposition efficiency (%)=[1−(concentration of ozone flowing out of the device)/(concentration of ozone flowing into the ozone decomposition unit)]×100 Equation 2

The heating may be performed for 10 minutes to 10 hours at the temperature of 80° C. to 250° C., while one cycle is set to a time period from a time point when the ozone decomposition efficiency represented by Equation 2 is reduced to less than 90% to a time point when the ozone decomposition efficiency is recovered to 90% or more.

The harmful gas may include organic-inorganic harmful gas including ethylene, ammonia, acetaldehyde, or a combination of these.

The harmful microorganisms may include fungi, E. coli, Pseudomonas aeruginosa, Staphylococcus, or a combination of these.

According to another aspect, provided is an air-cleaning system including the air-cleaning device.

According to another aspect, provided is an operation method of the air-cleaning device using the catalytic activity recovery method of a manganese oxide catalyst.

The operation method may be performed periodically by repeating the air purification process and the heating process for recovering the catalyst activity.

### Advantageous Effects of Invention

A catalytic activity recovery method of a manganese oxide catalyst according to the present disclosure includes recovering the initial activity of the manganese oxide catalyst by heating a manganese oxide catalyst, which is used to decompose ozone and of which activity is thus reduced by 10% or more compared to the initial activity thereof, at the temperature of 80° C. to 250° C. in the air atmosphere. Once subjected to the recovering process, the ozone decomposition efficiency of the manganese oxide catalyst, which is represented by Equation 1, can be recovered to 90% or more of the initial ozone decomposition efficiency. The air-cleaning device and air-cleaning system, which include these manganese oxide catalysts, can show improved lifespan characteristics, that is, harmful gas including ethylene and harmful microorganisms can be removed for a long period of time.

### BRIEF DESCRIPTION OF DRAWINGS

FIG. 1 shows a schematic diagram of an ozone decomposition unit of an air-cleaning device according to an embodiment.

FIG. 2 shows a schematic diagram of an air-cleaning device according to an embodiment.

FIG. 3 shows a schematic diagram of an air-cleaning device according to another embodiment.

FIG. 4 shows a graph of ozone decomposition efficiency (%) when an ozone decomposition catalyst structure prepared according to Preparation Example 2 is heated to 150° C.

FIG. 5 shows a graph of ozone decomposition efficiency (%) when an ozone decomposition catalyst structure prepared according to Preparation Example 2 is heated to 80° C.

FIG. 6 shows evaluation results of the ozone decomposition efficiency for an air-cleaning device manufactured according to Example 1.

FIG. 7 shows evaluation results of the ozone decomposition efficiency for an air-cleaning device manufactured according to Comparative Example 1.

### MODE FOR THE INVENTION

Referring to the drawings attached hereto, a catalytic activity recovery method of a manganese oxide catalyst according to an example embodiment, an air-cleaning device using the same, an air-cleaning system including the air-cleaning device, and an operation method of the air-cleaning device by using the manganese oxide catalyst will be described in detail. The following embodiments are provided as an example, and do not limit the present disclosure, and the present disclosure is defined only by the claims to be described later. In addition, in this specification and the drawings, elements having substantially the same functions are denoted by the same reference numeral, and redundant explanations thereof will be omitted.

Unless defined otherwise, all technical and scientific terms used herein have the same meaning as commonly understood by one of ordinary skill in the art to which the present disclosure belongs. In case of conflicts in the meanings, the definition in the present specification, including definitions, will be preferred.

Although methods and materials similar or equivalent to those described herein may be used in the practice or testing of the present invention, suitable methods and materials are described herein. Singular expressions include plural expressions unless the context clearly indicates otherwise.

In the present specification, terms such as “include” or “have” are intended to indicate the existence of features, numbers, processes, operations, components, parts, components, materials, or combinations thereof described in the specification. It is to be understood that the possibility of the presence or addition of one or more other features, numbers, processes, operations, components, parts, components, materials, or combinations thereof is not preliminarily excluded.

The term “combinations thereof” used herein refers to a mixture, alloy or combination with one or more of the described components.

The term “and/or” used herein refers to any combination or all combinations of at least one constituent element of a list. The term “or” used herein refers to “and/or”. The terms “at least one type”, “one or more types”, or “one or more” before components may supplement the list of all constituent elements, and may not supplement individual constituent elements of the recited list.

The term “aspect ratio” used herein refers to “a ratio of a shorter length (or width) to a longer length” or “a ratio of diameter to length” according to the shape, unless defined otherwise.

The term “diameter” or “average diameter (D50)” refers to a diameter or average diameter (D50) based on the assumption that the shape is a sphere or a shape close to the sphere, unless specified otherwise. However, when the shape is not a sphere or a shape close to the sphere, the length (width) of the shorter axis is defined as a diameter.

The term “periodical heating” refers to repeatedly performing decomposing or removing ozone by an ozone decomposition catalyst, and heating the ozone decomposition catalyst for catalytic activity recovery by one cycle.

The term “air cleaning” used herein refers to an operation process of the air-cleaning device in which an ozone generating unit is operated to perform an air-cleaning function. However, when operated for the purpose of cleaning ozone existing outside the air-cleaning device, the ozone generating unit does not work.

The term “heating process” used herein refers to an operation process of the air-cleaning device in which a (nano) manganese oxide catalyst is heated for the purpose of recovery of activity.

In the drawings, the thickness is enlarged or reduced in order to clearly express various layers and regions. Like reference numerals denote like elements throughout the specification. Throughout the specification, when a component such as a layer, film, region, plate, or the like is described to be “on” or “above” another component, this includes not only the case in which one component is directly on another component, but also the case in which an intervening layer is placed between the components. Throughout the specification, terms such as first and second may be used to describe various elements, but the elements should not be limited by the terms. The terms are used only for the purpose of distinguishing one component from another.

Ozone is a substance with strong oxidizing power, and oxidizes and decomposes organic molecules and has sterilizing power against microorganisms. Although ozone is used to clean air based on the principle, when the ozone is released in the gas form into the space, the ozone may harm the health of people located in the space, and may cause oxidative damage on other substances exiting inside the space.

Various substances have been reported as catalysts capable of decomposing ozone, and manganese oxide-based substances are known to be effective for the decomposition of ozone. However, ozone decomposition catalysts are easily damaged when exposed to ozone, and thus, ozone decomposition efficiency is decreased. This is because, due to strong oxidizing power of ozone, the catalyst material is damaged. Therefore, the operation time of tools, instruments, devices, etc. using ozone decomposition catalysts is short.

The present disclosure proposes a catalytic activity recovery method of manganese oxide catalyst from among ozone decomposition catalysts.

A catalytic activity recovery method of a manganese oxide catalyst according to an embodiment may include recovering the initial activity of the manganese oxide catalyst by heating a manganese oxide catalyst which has been used to decompose ozone and of which activity is thus reduced by 10% or more compared to the initial ozone decomposition efficiency thereof, at the temperature of 80° C. to 250° C., so as to recover an ozone decomposition efficiency represented by Equation 1 to 90% or more of the initial ozone decomposition efficiency:

Ozone decomposition efficiency (%)=[1−(concentration of ozone flowing out of the reactor)/(concentration of ozone flowing into the reactor)]×100 Equation 1

For example, the catalytic activity recovery method may include recovering the ozone decomposition efficiency represented by Equation 1 to 95% or more of the initial ozone decomposition efficiency. Over time, the manganese oxide catalyst used to decompose ozone may have less active sites. A catalytic activity recovery method of a manganese oxide catalyst according to an embodiment may include recovering the initial activity of the manganese oxide catalyst by heating a manganese oxide catalyst which has been used to decompose ozone and of which activity is thus reduced by 10% or more compared to the initial ozone decomposition efficiency thereof due to reduced active sites, at the temperature of 80° C. to 250° C., so as to recover an ozone decomposition efficiency represented by Equation 1 to 90% or more of the initial ozone decomposition efficiency.

The catalytic activity recovery method may include recovering the ozone decomposition efficiency represented by Equation 1 to 95% or more of the initial ozone decomposition efficiency.

The heating may include periodical heating for 10 minutes to 10 hours in a temperature range of 80° C. to 250° C. while one cycle is set to a time period from a time point when the ozone decomposition efficiency represented by Equation 1 is reduced to less than 90% of the initial ozone decomposition efficiency to a time point when the ozone decomposition efficiency is recovered to 90% or more of the initial ozone decomposition efficiency.

The heating may include periodical heating at a heating rate of 1° C./min to 10° C./min for 10 minutes to 10 hours in a temperature range of 80° C. to 250° C. while one cycle is set to a time period from a time point when the ozone decomposition efficiency represented by Equation 1 is reduced to less than 90% of the initial ozone decomposition efficiency to a time point when the ozone decomposition efficiency is recovered to 90% or more of the initial ozone decomposition efficiency.

The heating may be performed using a planar heating element, an electric resistance heating device, a heating furnace, a heating oven, infrared-ray heating, or a microwave generator.

The planar heating element may include a polymer resin, a carbon material, a metal material, a ceramic material, or a composite thereof. Examples of the polymer resin include polyester resin, unsaturated polyester resin, polycarbonate resin, polyacrylic resin, polyvinylidene fluoride, polypyrrole, epoxy resin, polyimide resin, polyurethane resin, nylon resin, polyacrylonitrile, polyvinylpyrrolidone, or a mixture thereof. Examples of the carbon material include graphite, carbon black, graphene, carbon nanotubes, carbon nanofibers, graphene oxide, reduced graphene oxide, or fullerene. Examples of the metal material include Ag, Au, Pt, Cu, Sn, Ni, Fe, or Al. However, the present disclosure is not limited thereto, and any material that is used for a surface heating element in the related art may be used. The planar heating element may be a sheet, a film, a pad, or fiber.

The planar heating element may further include a nonwoven fabric layer on a surface.

The manganese oxide catalyst may include at least one of α-MnO₂, β-MnO₂, γ-MnO₂, δ-MnO₂, or amorphous MnO₂. In an embodiment, the manganese oxide catalyst may contain α-MnO₂.

The manganese oxide catalyst may have a shape selected from a sphere shape, an oval shape, a rod shape, a fiber shape, a sea-urchin shape, a flower shape, or a sheet shape.

The manganese oxide catalyst may include at least one of α-MnO₂ or β-MnO₂, and the α-MnO₂ or β-MnO₂ has a nanorod shape, a nanofiber shape, a nano sea-urchin shape, or a nanoflower shape, and has an aspect ratio of 1:5 to 1:1000.

The manganese oxide catalyst may be α-MnO₂, and the α-MnO₂ may be in the shape of a nanorod or a nano sea-urchin, and the aspect ratio thereof may be 1:100.

The manganese oxide catalyst may further include a manganese oxide doped with a transition metal of 0.01 wt % to 50 wt % based on the total weight of the manganese oxide catalyst. In an embodiment, the manganese oxide catalyst may further include a manganese oxide doped with transition metal of 0.01 wt % to 45 wt %, transition metal of 0.01 wt % to 40 wt %, transition metal of 0.01 wt % to 35 wt %, transition metal of 0.01 wt % to 30 wt %, based on the total weight of the manganese oxide catalyst. Examples of the doped transition metal include copper, cerium, iron, cobalt, or nickel. However, the transition metal is not limited thereto, and any transition metal that is available in the related art may be used for the doping. A manganese oxide doped with such a transition metal may effectively maintain ozone decomposition catalytic activities for a long time even under a high humidity environment.

The manganese oxide catalyst may further include at least one selected from metal oxide, silicon oxide, carbon nanotubes, activated carbon, graphene, or graphene oxide. Examples of the metal oxide include copper oxide, cerium oxide, cobalt oxide, nickel oxide, or aluminum oxide. However, the metal oxide is not limited thereto, and any metal oxide that is available in the related art may be used. Examples of the carbon nanotubes may include single-walled carbon nanotubes (SWCNT), multi-walled carbon nanotubes (MWCNT), or a combination of these. Such a manganese oxide further including at least one selected from metal oxide, silicon oxide, carbon nanotubes, activated carbon, graphene, or graphene oxide may have higher ozone decomposition catalytic activities.

An air-cleaning device according to another an embodiment may use the catalytic activity recovery method of the manganese oxide catalyst described above.

The air-cleaning device for reducing harmful gases including ethylene and harmful microorganisms, the air-cleaning device includes: an ozone generating unit in which an ozone generating unit driven by electric energy to generate ozone is located; an ozone decomposition unit in which at least one ozone decomposition catalyst structure for decomposing ozone generated by the ozone generating unit is located; and a heating member for heating the ozone decomposition catalyst structure, wherein the ozone decomposition catalyst structure includes a support and nano manganese oxide located on at least a portion of the surface and inside of the support, The heating member is located within the ozone decomposition unit or is located separate from the ozone decomposition unit, and allows inflow and outflow of air in one direction.

The support may be a ceramic material, a metal material, or a combination of these, in the form of a monolith or a foam. Examples of the metal material include stainless steel or aluminum. However, the metal oxide is not limited thereto, and any metal material that is available in the related art may be used.

In an embodiment, the support may be a porous support. In an embodiment, the support may be a porous inorganic-material support. In an embodiment, the support may be a monolith.

In an embodiment, the porous inorganic-material support may include a porous ceramic material containing 50% or more of MgO, SiO₂, and Al₂O₃. The porous ceramic material may have a ceramic honeycomb structure. The porous ceramic material may have, per inch, about 100 to about 500, for example, about 200 to about 500, for example, about 300 to about 400 square cells. In the porous ceramic material, air or the like may be introduced through the square cells.

The porous ceramic material may increase a catalytic activity due to a high strength and a large specific surface area. In addition, the porous ceramic material has good ventilation and thus, may reduce pressure loss, and maintains the shape thereof even by external environments such as strong acids, high temperatures, and strong winds.

The porous ceramic material may have a cross section having various shapes such as a circular shape, an oval shape, a rectangular shape, or a square shape. The porous ceramic material may have the structure of a cylinder, a rectangular parallelepiped, or a cube, each having a height and diameter of several millimeters (mm) or hundreds of millimeters (mm). However, the porous ceramic material is not limited thereto, and various types of porous ceramic materials that can be used by those skilled in the art may be used.

The porous ceramic material may further include an alkali oxide component. Examples of the alkali oxide component include Li₂O, Na₂O, or K₂O. The porous ceramic material further including the alkali oxide component may maintain the shape of the ozone decomposition catalyst structure even at high temperatures without thermal deformation.

Such a support has a large specific surface area even compared to a support containing organic materials such as polybenzimidazole or polyamide, and may show excellent α-MnO₂ catalytic activity. In addition, the support may retain the shape thereof even by external environments such as strong acids, high temperature, and strong wind.

From among transition metal oxides, the nano manganese oxide actively generates reactive oxygen species required to oxidize harmful gases including ethylene by ozone at a low temperature of 100° C. or less. In an embodiment, reactive oxygen species are produced by decomposition of ozone. Compared to other transition metal oxides, the nano manganese oxide has oxygen vacancies enough to generate reactive oxygen species required for decomposition of ozone. Therefore, the nano manganese oxide has higher ozone decomposition activity compared to other transition metal oxides.

The nano manganese oxide may include at least one of α-MnO₂, β-MnO₂, γ-MnO₂, δ-MnO₂, or amorphous MnO₂. The nano manganese oxide may have a shape selected from a nanoparticle, a nanorod shape, a nanofiber shape, a nano sea-urchin shape, or a nanoflower shape.

In an embodiment, the nano manganese oxide may include at least one of α-MnO₂ or β-MnO₂, and the α-MnO₂ or β-MnO₂ may have a nanorod shape, a nanofiber shape, and a nano sea-urchin shape, or a nanoflower shape, and an aspect ratio of 1:5 to 1:1000. In an embodiment, the aspect ratio of a nanorod shape, a nanofiber shape, and a nano sea-urchin shape may be from 1:5 to 1:100, from 1:5 to 1:90, from 1:5 to 1:80, from 1:5 to 1:70, from 1:5 to 1:60, from 1:5 to 1:50, from 1:5 to 1:40, or from 1:5 to 1:30.

In an embodiment, the nano manganese oxide may be α-MnO₂, and the α-MnO₂ may have a nanorod shape or a nano sea-urchin shape. Since α-MnO₂ has more oxygen vacancies to generate reactive oxygen species required for the ozone decomposition, α-MnO₂ has excellent ozone decomposition catalytic activity compared to manganese oxide having other crystal structures.

For example, the manufacturing method of α-MnO₂ is as follows. At room temperature, a manganese chloride (MnCl₂·4H₂O) aqueous solution, a manganese acetate (Mn(CH₃COO)₂·4H₂O) aqueous solution, or a manganese sulfate (MnSO₄·5H₂O) aqueous solution, which are used as a starting material, is reacted with a predetermined equivalent amount of KMnO₄ to precipitate MnO₂, which is then reacted in a hydrothermal reactor at a temperature of 180° C. for 12 hours. In addition, in order to increase the yield or/and purity of the α-MnO₂ catalyst, the manufacturing method may be performed several times.

When the aspect ratio of the nanorod shape, the nanofiber shape, or the nano sea-urchin shape is within the range, the specific surface area is large and thus, excellent catalytic activity may be obtained, and the coating may be performed in a binder-free form.

In an embodiment, the nano manganese oxide may be δ-MnO₂, the δ-MnO₂ may have the nanoflower shape or the nanosheet form, and may have the thickness of 5 nm to 100 nm. In an embodiment, the thickness of the nano manganese oxide may be from 5 nm to 90 nm, from 5 nm to 80 nm, from 5 nm to 70 nm, from 5 nm to 60 nm, or from 5 nm to 50 nm. Only within these ranges, the nano manganese oxide has a large specific surface area with respect to the weight thereof, and thus, excellent catalytic activity may be obtained.

In an embodiment, the nano manganese oxide may be a crystalline MnO₂ or amorphous MnO₂ nanoparticle, and the nanoparticle may have a diameter of 1 nm to 500 nm. The diameter range of the nanoparticles refers to the diameter range of the short-axis length (or width). In an embodiment, the diameter of the nanoparticles may be from 1 nm to 450 nm, from 1 nm to 400 nm, from 1 nm to 350 nm, from 1 nm to 300 nm, from 1 nm to 250 nm, or from 1 nm to 200 nm. Nano manganese oxide may be easily coated using a coating solution containing nano manganese oxide having these diameter ranges, and, after the coating, the nano manganese oxide is not separated from the support and catalytic activity may be maintained high.

The nano manganese oxide may further include nano manganese oxide doped with a transition metal in an amount of 0.01 wt % to 50 wt % based on the total weight of the nano manganese oxide. In an embodiment, the nano manganese oxide may further include nano manganese oxide doped with 0.01 wt % to 45 wt % of transition metal, 0.01 wt % to 40 wt % of transition metal, 0.01 wt % to 35 wt % of transition metal, or 0.01 wt % to 30 wt % of transition metal, based on the total weight of the nano manganese oxide. Examples of the doped transition metal include copper, cerium, iron, cobalt, and nickel. However, the transition metal is not limited thereto, and any transition metal that is available in the related art may be used for the doping. The nano manganese oxide doped with such a transition metal may effectively maintain the catalytic activity of ozone decomposition for a long time even under a high humidity environment.

The nano manganese oxide may further include at least one selected from metal oxide, silicon oxide, carbon nanotubes, activated carbon, graphene, or graphene oxide. Examples of the metal oxide include copper oxide, cerium oxide, cobalt oxide, nickel oxide, or aluminum oxide. However, the metal oxide is not limited thereto, and any transition metal oxide that is available in the related art may be used. Examples of the carbon nanotubes may include single-walled carbon nanotubes (SWCNT), multiwalled carbon nanotubes (MWCNT), or a combination of these. The nano manganese oxide further including at least one selected from metal oxide, silicon oxide, carbon nanotubes, activated carbon, graphene, or graphene oxide has high affinity for harmful gases including ethylene, and thus can effectively adsorb the harmful gases. Therefore, the nano manganese oxide may have a higher catalytic activity of ozone decomposition.

The ozone decomposition catalyst structure may be a binder-free structure. A sufficient amount of a binder is required to coat (nano) manganese oxide on an organic material support such as a commonly used fiber aggregate, and the catalytic activity of ozone decomposition may be reduced. In addition, since organic material supports such as fiber aggregates have flexible properties, the shapes thereof are changed due to external environments such as strong acids, high temperatures, and strong winds. Accordingly, there is a need to provide a separate design to fix the supports. The ozone decomposition catalyst structure may be fixed on the pores and surfaces inside the support without a binder, so the catalytic activity of ozone decomposition can be further increased.

At least one side of the ozone decomposition catalyst structure may be in contact with a sheet, film, pad, or foil of a thermally conductive material. Examples of the thermally conductive material include: metals such as Al; metalloids such as Si; or complexes thereof, but are not limited thereto.

The heating member may be one of a planar heating element, an electric resistance heating device, or a microwave generating unit.

The planar heating element may include a polymer resin, a carbon material, or a composite thereof, which may be in the form of a sheet or a film. Examples of the polymer resin include polyester resin, unsaturated polyester resin, polycarbonate resin, polyacrylic resin, polyvinylidene fluoride, polypyrrole, epoxy resin, polyimide resin, polyurethane resin, nylon resin, polyacrylonitrile, polyvinylpyrrolidone, or a mixture thereof. Examples of the carbon material include graphite, carbon black, graphene, carbon nanotubes, carbon nanofibers, graphene oxide, reduced graphene oxide, or fullerene. However, the present disclosure is not limited thereto, and any material that is used for a surface heating element in the related art may be used. In an embodiment, the planar heating element may be a carbon material sheet having a power of 50 Watts or less.

The planar heating element may be brought in contact with a heat insulating material. Examples of the heat insulating material include silica, but are not limited thereto, and all heat insulating materials available in the art may be used.

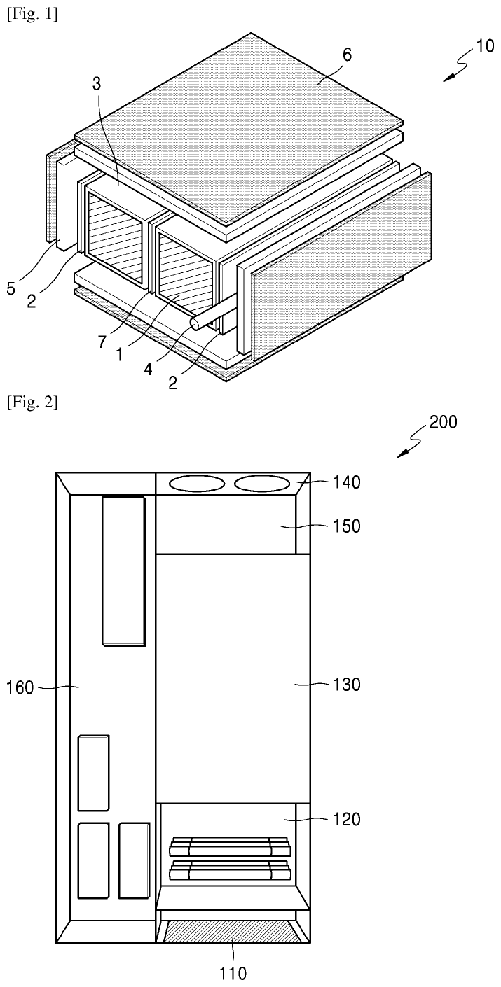

FIG. 1 shows a schematic diagram of an ozone decomposition unit 10 of an air-cleaning device according to an embodiment.

As illustrated in FIG. 1, the ozone decomposition unit 10 of the air-cleaning device includes an ozone decomposition catalyst structure 1 and an ozone decomposition catalyst structure 1 including a thermally conductive material foil 3 surrounding the surface thereof. A first planar heating element 2 and a second planar heating element 7 are arranged between adjacent ozone decomposition catalyst structures 1 and between the ozone decomposition catalyst structure 1 and a case 6, respectively. The side surfaces of the second planar heating element 7 and ozone decomposition catalyst structure 1 are surrounded by a heat insulating material 5 on the side thereof, and a temperature sensor 4 is located between the second planar heating element 7 and the case 6.

FIG. 2 shows a schematic diagram of an air-cleaning device 200 according to an embodiment.

Referring to FIG. 2, the air-cleaning device 200 according to an embodiment includes an air inlet 110, an ozone generating unit 120, an ozone decomposition unit 130, an air outlet 140, a fan 150, and an electric device unit 160.

The air inlet 110 is an area through which air is introduced from the outside.

The ozone generating unit 120 may include one or more ozone generators of a vacuum ultraviolet lamp, a corona discharge ozone generator, or a cold plasma ozone generator.

In an embodiment, the ozone generating unit 120 may be a vacuum ultraviolet lamp or a corona discharge ozone generator. In an embodiment, the vacuum ultraviolet lamp may be a UV-C lamp. When the UV-C lamp is used with an output of 8 W or more within the ranges of the wavelength ratio, the performance of reducing or removing harmful gases including ethylene and harmful microorganisms, which are difficult to be decomposed by a photocatalyst, may be improved. In order to further improve the performance of reducing or removing harmful gases including ethylene and harmful microorganisms, the number, voltage, current, or output of UV-C lamps may be appropriately adjusted, for example, increased. The UV-C lamp may be surrounded by one or more photocatalytic structures. The photocatalytic structure may include a substrate and a TiO₂ photocatalyst arranged on the substrate. The example of the substrate is not limited. In an embodiment, the substrate may be, for example, a TiO₂ photocatalyst-coated a stainless steel mesh having a three-dimensional network structure or a TiO₂ photocatalyst-coated transparent glass tube. In this case, the coating method is not limited, but, for example, dip coating may be used.

In addition, the ozone generating unit 120 may further include an electric generator, an electric energy storage unit, and a charge controller. The electric energy storage unit is electrically connected to an electric generator and stores the electric energy produced therefrom. The charge controller is configured to be combined with the electric generator and the electric energy storage unit and is configured to control the charging and discharging of electricity in the electric generator and the electric energy storage unit. An example of the electric generator may be a horizontal axis turbine, and an example of the electric energy storage unit may be a battery. The air inlet 110 and the ozone generating unit 120 may be physically connected to each other through a pipe (not illustrated) or an air passage that guides the air flow to the ozone generating unit 120 (not illustrated). Alternatively, the air inlet 110 may be fixedly arranged on one surface of the ozone generating unit 120.

The ozone decomposition unit 10 described above may be located in the ozone decomposition unit 130, and the ozone decomposition unit 10 may include an ozone decomposition catalyst structure and a heating element.

The air outlet 140 is an area through which internal air is discharged to the outside.

The air-cleaning device 200 enables the inflow and outflow of air in one direction.

A fan 150 may be provided in at least one of the air inlet 110 and the air outlet 140. In an embodiment, the fan 150 may be fixedly arranged on the air inlet 110 and the air outlet 140 or in a separate area.

The air-cleaning device 200 may additionally include a blower, a compressor, or a pump.

The air-cleaning device 200 periodically heats a nano manganese oxide catalyst which has been used to decompose ozone and of which activity is thus reduced by 10% or more of the initial ozone decomposition efficiency thereof, at the temperature of 80° C. to 250° C., from a time point when the ozone decomposition efficiency represented by Equation 2 is reduced to less than 90% of the initial ozone decomposition efficiency to a time point when the ozone decomposition efficiency is recovered to 90% or more compared to the initial ozone decomposition efficiency, to recover the catalytic activity, thereby maintaining the ozone decomposition efficiency represented by Equation 2 to be 90% or more based on the initial ozone decomposition efficiency:

Ozone decomposition efficiency (%)=[1−(concentration of ozone flowing out of the device)/(concentration of ozone flowing into the ozone decomposition unit)]×100 Equation 2

The heating may be performed for 10 minutes to 10 hours at the temperature of 80° C. to 250° C., while one cycle is set to a time period from a time point when the ozone decomposition efficiency represented by Equation 2 is reduced to less than 90% to a time point when the ozone decomposition efficiency is recovered to 90% or more.

Through the periodic heating, the activity of the nano manganese oxide catalyst and the ozone decomposition efficiency may have 90% or more. Through the periodic heating, the activity of the nano manganese oxide catalyst which has been used to decompose ozone and of which activity is reduced, may be recovered.

The harmful gas may include organic-inorganic harmful gas including ethylene, ammonia, acetaldehyde, or a combination thereof. The harmful microorganisms may include fungi, E. coli, Pseudomonas aeruginosa, Staphylococcus, or a combination of these.

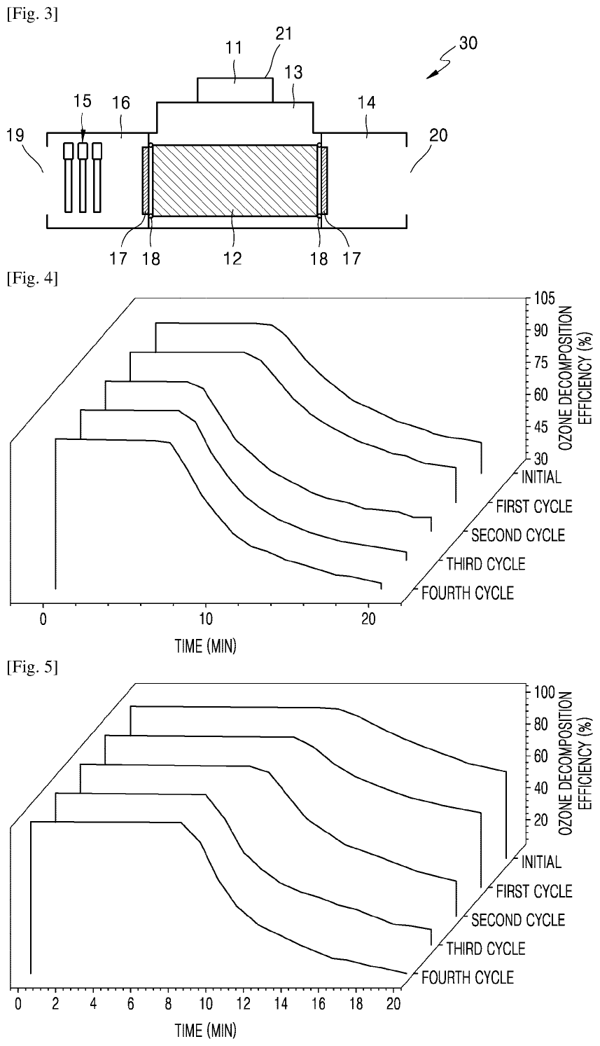

FIG. 3 shows a schematic diagram of an air-cleaning device 30 according to an embodiment.

As illustrated in FIG. 3, the air-cleaning device 30 according to an embodiment includes, in a case 21, an air inlet 19, a first reaction chamber 16 in which an ozone generating unit 15 is located, a second reaction chamber 13 in which a ozone decomposition catalyst structure 12 is located, a third reaction chamber 11 in which a heating part is located, and an air outlet 14 in which a fan 20 is provided.

The heating part may include a microwave generator. In an embodiment, the microwave generator may have a power of 500 Watt or less. The ozone decomposition catalyst structure 12 may be heated by irradiation by a microwave generator such as a magnetron in the third reaction chamber 11. Irradiation by the microwave generator may heat the ozone decomposition catalyst structure 12 without leaking to the outside by a metal grid 17 and the partition walls of the first reaction chamber 16 and the air outlet 14. The ozone decomposition catalyst structure 12 may be fixed to the partition walls of the first reaction chamber 16 and the air outlet 14 by an airtight gasket 18.

The air inlet 19, the ozone generating unit 15, the ozone decomposition catalyst structure 12, the fan 20, and the like are the same as described above, and thus a detailed description thereof will be omitted.

An air-cleaning system according to an embodiment may include the air-cleaning device described above. The air-cleaning system may further include a sensor, or a temperature controller, when needed.

The operation method of the air-cleaning device according to another an embodiment may use the catalytic activity recovery method of the manganese oxide catalyst described above.

The operation method may be performed periodically by repeating the air purification process and the heating process for recovering the catalyst activity. Hereinafter, Examples and Comparative Examples of the present disclosure will be described. However, the following example is only an example of present disclosure, and the present disclosure is not limited to the following examples.

### EXAMPLES

### Preparation Example 1: Preparation of Ozone Decomposition Catalyst Structure

13.1 g of MnCl₂-4H₂O and 21.7 g of KMnO₄ were added to 250 ml of water and stirred together to obtain a mixed solution. 250 mL of the mixed solution was heated to the temperature of 220° C. for 2 hours in a hydrothermal reactor, caused to react at a temperature of 220° C. for 12 hours, and then filtered. Then, the obtained precipitate was dried at a temperature of 100° C. for 2 hours to obtain a bulky α-MnO₂. A α-MnO₂-containing solution (a solid content of about 10%) in which the bulky α-MnO₂ was dispersed in water, was milled. As a result, an α-MnO₂ dispersion containing ax-MnO₂ nanorods having an aspect ratio (diameter:length) of about 1:40 was obtained.

A porous cordierite monolith (Ceracomb Co., Ltd.) having the shape of rectangular parallelepiped, containing 50% or more of MgO, SiO₂, and Al₂O₃ components, and having the size of 100 mm×100 mm×400 mm was prepared. The porous cordierite monolith was dipped in the α-MnO₂ dispersion and then dried to prepare an ozone decomposition catalyst structure in which the α-MnO₂ nanorod is coated the inside and surface of the porous cordierite monolith.

At this time, the amount of the α-MnO₂ catalyst was 10 parts by weight based on 100 parts by weight of the porous cordierite monolith.

### Preparation Example 2: Preparation of Ozone Decomposition Catalyst Structure

An ozone decomposition catalyst structure was prepared in the same manner as in Preparation Example 1, except that a porous cordierite monolith having the cylinder shape and the size of 50 mm×50 mm (Ceracomb Co., Ltd.) was used instead of the porous cordierite monolith (Ceracomb Co., Ltd.) having the shape of rectangular parallelepiped, containing 50% or more of MgO, SiO₂, and Al₂O₃ components, and having the size of 100 mm×100 mm×400 mm to manufacture an ozone decomposition catalyst structure in which the amount of α-MnO₂ catalyst was 7 parts by weight based on 100 parts by weight of the porous cordierite monolith.

### Preparation Example 3: Manufacture of Ozone Decomposition Unit

An ozone decomposition unit 10 illustrated in FIG. 1 was manufactured.

Side surfaces of the ozone decomposition catalyst structure 1 prepared according to Preparation Example 1, not top and bottom surfaces thereof where pores are located, was surrounded by an aluminum foil 3, which is a thermally conductive material. Two of such an ozone decomposition catalyst structure 1 surrounded by the aluminum foil 3 were prepared. A carbon-material surface heating element sheet (manufactured by i-ONE FILM Co., Ltd.) 2 having a power of 50 Watt, as a first planar heating element, was arranged between the ozone decomposition catalyst structures 1. A carbon-material surface heating element sheet (manufactured by i-ONE FILM Co., Ltd.) 2 having a power of 30 Watt, as a second planar heating element, was arranged between the ozone decomposition catalyst structure 1 and the case 6. Side surfaces of the ozone decomposition catalyst structure 1 surrounded by the second planar heating element 7 and the aluminum foil 3 were surrounded by the heat insulating material formed of a silica material (25T, Banseuk Co., Ltd). A temperature sensor 4 was arranged between the second planar heating element 7 and the case 6, thereby completing the manufacture of the ozone decomposition unit 10.

### Comparative Preparation Example 1: Preparation of Ozone Decomposition Unit

Two ozone decomposition catalyst structures prepared according to Preparation Example 1 were provided inside a case.

### Example 1: Manufacture of Air-Cleaning Device

An air-cleaning device 200 as illustrated in FIG. 2 was manufactured as follows.

A stainless steel plate having a thickness of 1T (mm) was bent to manufacture a case 170. An ozone generating unit 120 having an air inlet (including a fan, 110) provided therein, was provided in the case 170. The ozone generating unit 120 was equipped with six UV-C lamps (manufactured by Light Sources Inc.). The ozone decomposition unit 130 according to Preparation Example 2 was provided above the ozone generating unit 120. The ozone generating unit 120 and the ozone decomposition unit 130 were connected via a connecting pipe (not illustrated), which was used as a passage for introducing the ozone-containing air generated from the ozone generating unit 120 into the ozone decomposition unit 130. An air outlet 140 was provided above the ozone decomposition unit 130, and through the air outlet 140, cleaned air was discharged. An axial fan (manufactured by ebm-papst Inc.) 150 was provided above the air outlet 140, thereby manufacturing the air-cleaning device 200.

In the air-cleaning device 200, the places where air could be accessed from the outside were limited to the air inlet 110 and the air outlet 140. The air-cleaning device 200 was configured to allow air to flow in one direction from the air inlet 110 to the ozone generating unit 120, the ozone decomposition unit 130, the air outlet 140, and the fan 150.

### Comparative Example 1: Manufacture of Air-Cleaning Device

An air-cleaning device was manufactured in the same manner as the air-cleaning device according to Example 1, except that an air-cleaning device was manufactured using the ozone decomposition unit according to Comparative Preparation Example 1.

### Evaluation Example 1: Catalytic Activity Recovery Evaluation

The ozone decomposition catalyst structure according to Preparation Example 2 was placed in a tubular flow reactor and exposed to air having an ozone concentration of 100±10 ppm and a humidity of 90±5% for 20 minutes at a flow rate of 18 L per minute. The concentration of ozone flowing into the reactor and the concentration of ozone flowing out of the reactor were each measured, and evaluated through ozone decomposition efficiency obtained by substituting the same in Equation 1 below. The inflow and outflow ozone concentrations were measured using an ozone analyzer based on ultraviolet absorption method (Model 202, 2B technology Inc.). The ozone decomposition catalyst structure which was used once evaluated, was heated by an electric heating oven at 150° C. or 80° C., with a heating rate of 5° C./min for 3 hours each, and cooled at room temperature, and then, placed in a tubular flow reactor and exposed to ozone-including air under the conditions and evaluated again as described above. The evaluation was performed for 5 times. The results are shown in Tables 1 and 2, and FIGS. 4 and 5.

Ozone decomposition efficiency (%)=[1−(concentration of ozone flowing out of the reactor)/(concentration of ozone flowing into the reactor)]×100 Equation 1

TABLE 1

| | Ozone decomposition efficiency (%) | | | | |
|---|---|---|---|---|---|
| | after heating at 150° C. | | | | |
| Time | | First | Second | Third | Fourth |
| (min) | Initial | cycle | cycle | cycle | cycle |
| 0 | 99.95 | 99.23 | 99.73 | 99.83 | 99.93 |
| 5 | 99.91 | 99.52 | 99.57 | 99.60 | 99.29 |
| 10 | 77.12 | 78.29 | 58.86 | 60.51 | 63.64 |
| 15 | 53.79 | 55.43 | 43.10 | 40.55 | 41.71 |
| 20 | 44.11 | 46.23 | 36.18 | 33.80 | 32.64 |

TABLE 2

| | Ozone decomposition efficiency (%) | | | | |
|---|---|---|---|---|---|
| | after heating at 100° C. | | | | |
| Time | | First | Second | Third | Fourth |
| (min) | Initial | cycle | cycle | cycle | cycle |
| 0 | 99.93 | 99.92 | 98.96 | 99.92 | 99.67 |
| 5 | 99.52 | 99.45 | 98.95 | 99.58 | 99.60 |
| 10 | 99.40 | 98.85 | 94.72 | 62.72 | 62.94 |
| 15 | 77.93 | 65.88 | 45.57 | 30.23 | 19.44 |
| 20 | 58.91 | 51.52 | 26.42 | 14.22 | 5.612 |

Referring to Tables 1 and 2 and FIGS. 4 and 5, it was confirmed that, when the ozone decomposition catalyst structure according to Preparation Example 2 was exposed to ozone for 20 minutes in the tubular flow reactor, the ozone decomposition efficiency was reduced over time and after heated in air, the ozone decomposition catalyst structure recovered the ozone decomposition efficiency thereof.

In Table 1 and FIG. 4, immediately after synthesis, the ozone decomposition catalyst structure according to Preparation Example 2 had an initial ozone decomposition efficiency (ozone decomposition efficiency at 0 min) of 99.95%, and the ozone decomposition efficiency was monotonically decreased with time to 44.11%. This is a decrease in activity by 44.13% (44.11/99.95) of the initial ozone decomposition efficiency. After being heated in an electric heating oven at 150° C. for 3 hours, the ozone decomposition efficiency of the same catalyst was 99.23%, which corresponds to the recovery of 99.97% of the initial ozone decomposition efficiency immediately after synthesis. Thereafter, when the same process was repeated, after 20 minutes, the ozone decomposition efficiency was decreased to about 30% of the initial ozone decomposition efficiency immediately after synthesis. However, in the case of heating at a temperature of 150° C., the initial ozone decomposition efficiency was 99% of the initial ozone decomposition efficiency immediately after synthesis.

In Table 2 and FIG. 5, immediately after synthesis, the ozone decomposition catalyst structure according to Preparation Example 2 had an initial ozone decomposition efficiency (ozone decomposition efficiency at 0 min) of 99.93%, and the ozone decomposition efficiency was monotonically decreased with time to 58.91%. This is a decrease in activity by 58.95% (58.91/99.93) of the initial ozone decomposition efficiency. After being heated in an electric heating oven at 80° C. for 3 hours, the ozone decomposition efficiency of the same catalyst was 99.92%, which corresponds to the recovery of 99.98% of the initial ozone decomposition efficiency immediately after synthesis. Thereafter, when the same process was repeated, after 20 minutes, the ozone decomposition efficiency was decreased to about 5% to 50% of the initial ozone decomposition efficiency immediately after synthesis. However, in the case of heating at a temperature of 80° C., the initial ozone decomposition efficiency was 98% of the initial ozone decomposition efficiency immediately after synthesis.

### Evaluation Example 2: Evaluation of Ozone Decomposition Efficiency

The ozone decomposition efficiency was evaluated for the air-cleaning devices according to Example 1 and Comparative Example 1. The ozone decomposition efficiency was evaluated in the following manner.

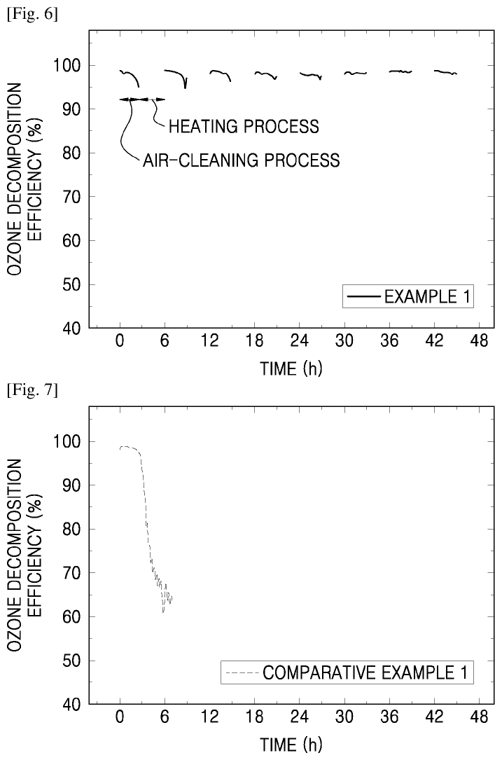

The air-cleaning devices according to Example 1 and Comparative Example 1 were each placed in a chamber of 2 m×2 m×2 m. The chamber was filled with air and the chamber environment was maintained at a temperature of 3° C. and at the relative humidity of 80% to 95%. The air-cleaning device according to Example 1 temporarily stopped the operation of an ozone generating unit, an air inlet, and an air outlet before the ozone decomposition efficiency was reduced to 95% or less in the air-cleaning process, and was subjected to a heating process at a temperature of 150° C. with the heating rate of 2.5° C./min for about 3 hours. These processes were repeatedly performed. The air-cleaning device according to Comparative Example 1 was continuously operated. The concentration of ozone which flew into a device and flew out toward the outside according to an operation time of each air-cleaning device, was measured. The inflow and outflow ozone concentrations were measured using an ozone analyzer based on ultraviolet absorption method (Model 202, manufactured by 2B technology Inc.). The ozone decomposition efficiency was calculated by substituting the ozone concentrations measured above into Equation 2 below. The results are shown in FIGS. 6 and 7.

Ozone decomposition efficiency (%)=[1−(concentration of ozone flowing out of the device)/(concentration of ozone flowing into the ozone decomposition unit)]×100 Equation 2

As shown in FIG. 6, the air-cleaning device according to Example 1 maintained an ozone decomposition efficiency of 95% or more for 45 hours. As illustrated in FIG. 7, in the case of the air-cleaning device according to Comparative Example 1, the ozone decomposition efficiency was continuously reduced by about 35% for up to 6 hours. From these results, it can be seen that the nano manganese oxide catalyst of the ozone decomposition catalyst structure included in the air-cleaning device according to Example 1 was operated while maintaining the catalytic activity of 95% or more of the initial activity for 45 hours.

In the above, embodiments have been described in detail with reference to the accompanying drawings, but the present disclosure is not limited to the related examples. To those with ordinary knowledge in the technical field to which the present disclosure belongs, it is clear that their thoughts affect various changes or modifications within the scope of the technical concept described in the claims. Even these changes and modifications are understood to belong to the technical range of the present disclosure.

1. A catalytic activity recovery method of a manganese oxide catalyst, the catalytic activity recovery method comprising:
recovering an ozone decomposition efficiency represented by Equation 1 to 90% or more of an initial ozone decomposition efficiency by heating, in an air atmosphere, a manganese oxide catalyst which has been used to decompose ozone and of which activity is thus reduced by 10% or more of the initial ozone decomposition efficiency thereof, at a temperature from 80° C. to 250° C.:
Ozone decomposition efficiency (%)=[1−(concentration of ozone flowing out of the reactor)/(concentration of ozone flowing into the reactor)]×100. Equation 1

2. (canceled)

3. The catalytic activity recovery method of claim 1, wherein
the heating comprises periodical heating for 10 minutes to 10 hours in a temperature range of 80° C. to 250° C. while one cycle is set to a time period from a time point when the ozone decomposition efficiency represented by Equation 1 is reduced to less than 90% of the initial ozone decomposition efficiency to a time point when the ozone decomposition efficiency is recovered to 90% or more of the initial ozone decomposition efficiency.

4. The catalytic activity recovery method of claim 3, wherein
the heating comprises periodical heating at a heating rate of 1° C./min to 10° C./min for 10 minutes to 10 hours in a temperature range of 80° C. to 250° C. while one cycle is set to a time period from a time point when the ozone decomposition efficiency represented by Equation 1 is reduced to less than 90% of the initial ozone decomposition efficiency to a time point when the ozone decomposition efficiency is restored to 90% or more of the initial ozone decomposition efficiency.

5. The catalytic activity recovery method of claim 1, wherein
the heating is performed using one of a planar heating element, an electric resistance heating device, a heating furnace, a heating oven, infrared-ray heating, or a microwave generator.

6. The catalytic activity recovery method of claim 5, wherein
the planar heating element comprises a polymer resin, a carbon material, a metal material, a ceramic material, or a composite of these.

7. The catalytic activity recovery method of claim 1, wherein
the manganese oxide catalyst comprises a nano manganese oxide including at least one of α-MnO₂, β-MnO₂, γ-MnO₂, δ-MnO₂, or amorphous MnO₂.

8. The catalytic activity recovery method of claim 1, wherein
the manganese oxide catalyst has a shape selected from a sphere shape, an oval shape, a rod shape, a fiber shape, a sea-urchin shape, a flower shape, or a sheet shape.

9. The catalytic activity recovery method of claim 1, wherein
the manganese oxide catalyst is at least one of α-MnO₂ or β-MnO₂, and
α-MnO₂ or β-MnO₂ has a nanorod shape, a nanofiber shape, a nano sea-urchin shape, or a nanoflower shape, and has the aspect ratio of 1:5 to 1:1000.

10. The catalytic activity recovery method of claim 1, wherein
the manganese oxide catalyst is α-MnO₂,
α-MnO₂ is a nanorod or nano sea-urchin shape, and has the aspect ratio of 1:100.

11. (canceled)

12. The catalytic activity recovery method of claim 1, wherein
the manganese oxide catalyst further comprises at least one selected from metal oxide, silicon oxide, carbon nanotubes, activated carbon, graphene, or graphene oxide.

13. An air-cleaning device using the catalytic activity recovery method of the manganese oxide catalyst of claim 1.

14. The air-cleaning device of claim 13, wherein
the air-cleaning device for reduce harmful gases including ethylene and harmful microorganisms and comprises:
an ozone generating unit in which an ozone generating unit driven by electric energy to generate ozone is located;
an ozone decomposition unit in which at least one ozone decomposition catalyst structure for decomposing ozone generated by the ozone generating unit is located; and
a heating member for heating the ozone decomposition catalyst structure,
the ozone decomposition catalyst structure includes a support and nano manganese oxide located on at least a portion of the surface and inside of the support,
the heating member is located within the ozone decomposition unit or located separate from the ozone decomposition unit, and
air is able to flow into and flow out in one direction.

15. The air-cleaning device of claim 14, wherein
the support is a ceramic material, a metal material, or a combination of these, in the form of a monolith or a foam.

16. (canceled)

17. The air-cleaning device of claim 14, wherein
the manganese oxide includes at least one of α-MnO₂ or β-MnO₂, and
α-MnO₂ or β-MnO₂ has a nanorod shape, a nanofiber shape, or a nano sea-urchin shape, and an aspect ratio of 1:5 to 1:1000.

18. (canceled)

19. The air-cleaning device of claim 14, wherein
at least one side of the ozone decomposition catalyst structure is in contact with a sheet, film, pad, or foil of a thermally conductive material.

20. The air-cleaning device of claim 14, wherein
the heating member is one of a surface heating element, an electric resistance heating device, or a microwave generator.

21. The air-cleaning device of claim 20, wherein
the surface heating element comprises a polymer resin, a carbon material, or a composite thereof, each in the form of a sheet or a film.

22. The air-cleaning device of claim 20, wherein
the surface heating element is in contact with a heat insulating material.

23. The air-cleaning device of claim 14, wherein
the ozone generating unit comprises one or more of a vacuum ultraviolet lamp, ultraviolet-C lamp and TiO₂, a corona discharge ozone generator, or a cold plasma ozone generator.

24. The air-cleaning device of claim 13, wherein
the air-cleaning device maintains an ozone decomposition efficiency represented by Equation 2 to be 90% or more of the initial ozone decomposition efficiency by periodical heating, at the temperature of 80° C. to 250° C., a nano manganese oxide catalyst which has been used to decompose ozone and of which activity is thus reduced by 10% or more compared to the initial ozone decomposition efficiency thereof, from a time point when the ozone decomposition efficiency represented by Equation 2 is reduced to less than 90% of the initial ozone decomposition efficiency to a time point when the ozone decomposition efficiency is recovered to 90% or more compared to the initial ozone decomposition efficiency, to recover the catalytic activity:
Ozone decomposition efficiency (%)=[1−(concentration of ozone flowing out of the device)/(concentration of ozone flowing into the ozone decomposition unit)]×100. Equation 2

25. The air-cleaning device of claim 24, wherein
the heating comprises periodical heating for 10 minutes to 10 hours in a temperature range of 80° C. to 250° C. while one cycle is set to a time period from a time point when the ozone decomposition efficiency represented by Equation 2 is reduced to less than 90% of the initial ozone decomposition efficiency to a time point when the ozone decomposition efficiency is recovered to 90% or more compared to the initial ozone decomposition efficiency.

26. (canceled)

27. (canceled)

28. An air-cleaning system comprising the air-cleaning device according to claim 13.

29. An operation method of an air-cleaning device using the catalytic activity recovery method of the manganese oxide catalyst of of claim 1.

30. The operation method of claim 29, wherein
the operation method periodically performing a cycle which includes an air-cleaning process and a heating process to recover catalytic activity.
